(19) United States
(12) Patent Application Publication
(10) Pub. No.: US 2006/0173298 A1
(43) Pub. Date: Aug. 3, 2006

(54) Intracardiac catheter and method of use

(76) Inventor: Kelly J Tucker, Villa Park, CA (US)

Correspondence Address: DLA PIPER RUDNICK GRAY CARY US, LLP, 4365 EXECUTIVE DRIVE, SUITE 1100, SAN DIEGO, CA, 92121-2133, US

(21) Appl. No.: 10/532,785

(22) PCT Filed: Oct. 29, 2003
(86) PCT No.: PCT/US03/34622

§ 371 Date: Oct. 17, 2005

Related U.S. Application Data
(60) Provisional application No. 60/422,366, filed on Oct. 29, 2002.

(51) Int. Cl. A61B 6/00

(52) U.S. Cl. 600/433

(57) ABSTRACT

The invention provides a cardiac balloon catheter and methods of using the same in the process of venography in the coronary sinus and pulmonary vein of the heart. The catheter has a flexible body containing a curved distal portion, at least three lumens running the length of the body each with a port of entry at the proximal end, an inflatable balloon located proximally of the curved distal portion of the body and port of exit of at least one of the lumens located at a distal end. Optionally, the catheter may contain up to ten pairs of bipolar electrodes. The invention provides methods for using the catheter to generate a geographic may of a venous structure of the heart and to generate an electrical map of the electrical conduction patterns of the heart for locating an aberrant electrical conduction pattern. Additionally, the invention catheter can be used in placement of pacemaker leads in the left ventricle for biventricular pacing of the heart.

### BACKGROUND OF THE INVENTION

1. Field of the Invention

The invention relates generally to cardiac catheters and methods of use and, more specifically, to use of the cardiac catheter in methods for mapping the anatomy or electrical conduction patterns of a cardiac chamber and surrounding connected vein branches.

2. Background Information

Subjects with irregularities in their circulatory system, due to a dysfunction of the heart, may require one or more of a number of procedures to remedy that dysfunction. Such procedures may include cardiac catheterization. In cardiac catheterization a catheter is inserted into a blood vessel through a distant percutaneous opening and fed through that blood vessel to the heart. By catheterization, either diagnostic or therapeutic procedures may be performed with minimal invasiveness. Diagnostic tests may include determination of pressure within the heart, the taking of blood samples, the taking of pictures of the blood in the chambers or vessels of the heart and electrophysiological testing, all of which can provide essential diagnostic information about the structure and function of the heart. Therapeutic applications of catheterization may include clearing blocked vessels, repair of vessels or valves and implantation of pacemakers.

The beating of the heart is regulated by electrical impulses from the sinoatrial (SA) node or sinus node. When the SA node ceases to function or functions improperly, the heartbeat can become irregular. A pacemaker, an artificial device that supplements or replaces the electrical impulses from the sinus node, may then be implanted to regulate the heartbeat.

Traditionally, pacemakers have been implanted to stimulate contraction of only the right ventricle or the right ventricle and the right atrium. A new method of pacing the heart is gaining popularity, in which the pacemaker contains leads to both the right and left ventricles. The impulses from the pacemaker stimulate simultaneous contraction of both ventricles, as occurs naturally in a normally functioning heart. This method of dual stimulation of the ventricles is called biventricular pacing. Generally, biventricular pacing is used to treat subjects with advanced congestive heart failure.

Current processes to implant the leads and, consequently, to implant the pacemakers for biventricular pacing are time-intensive and difficult. One step that is extremely difficult is the placement of a pacemaker lead into the coronary sinus or left ventricle. The difficulty of this task stems from the number of branches of the coronary sinus and the angles at which these branches project form the body of the coronary sinus. The coronary sinus is the final venous conduit draining venous (blue) blood from the coronary circulation into the right atrium. The os of the coronary sinus is located in the posterior right atrium adjacent to the interatrial septum. The coronary sinus extends from the os leftward in the atrioventricular groove around the left lateral border of the heart and ending in an anterior location. Biventricular pacing requires placement of the left ventricle pacing lead on the lateral wall of the left ventricle via venous tributaries of the coronary sinus.

Consequently, there is a need in the art for a more straightforward method of placing a pacemaker lead in the left ventricle and a catheter for facilitating the same.

Many types of catheters are known in the art that have been used for mapping the geography of the heart and placement of pacemakers. Most cardiac catheters require a sheath for insertion of the catheter. Typically, use of a catheter requires insertion of a needle, followed by insertion of a guidewire through a lumen in the needle, insertion of a sheath over the guidewire and then insertion of a catheter through the sheath. Certain cardiac catheters use electrical activation in order to obtain an electrical map of the heart. Still another type of cardiac catheter provides a geographical map of the heart, but cannot map the path of a coronary vessel until the catheter is physically inserted into the vessel and therefore does not provide means for mapping the branches of the venous system that lead into the main vessels.

Therefore, a need remains in the art for an instrument and a process that can map, not only the main vessels, but the coronary sinus, coronary sinus ostium, pulmonary vein, branches of the coronary sinus or pulmonary vein, or any other cardiac chamber or feature without necessitating travel throughout each vessel to be mapped to generate a geographical map of the branches of the venous system and without employing electrical activation techniques to map the electrical conduction patterns of desired areas of the heart.

### SUMMARY OF THE INVENTION

The present invention solves the problems in the art detailed above by providing a cardiac catheter and methods of its use for diagnostic and therapeutic purposes, including generation of geographic and electrical maps of the heart. The invention catheter may also be used in the placement and implantation of pacemakers, including in either the left or right ventricle.

In one embodiment, the invention provides balloon catheters with a flexible elongate body having a proximal portion and a distal portion, wherein the distal portion includes a curved portion terminating in a hooked portion at the extreme distal end, wherein curvature of the hooked portion is greater than curvature of the curved portion; at least three lumens running lengthwise throughout the body, each having a port of entry at a proximal end thereof; an inflatable balloon located on the exterior of the body with a distal end of the balloon terminating about 2 to 4 centimeters proximally of the curved portion of the distal portion of the body; and a port of exit located at a distal end of at least one of the lumens.

In another embodiment, the invention provides method for geographically mapping a venous structure of the heart by inserting a catheter into a vein of the heart; occluding the blood flow in the vein; injecting a radiographic medium into the vein, wherein the occlusion of the blood flow causes retrograde flow of the blood and the dye into the surrounding connected venous structure of the vein, including branches of the vein; and imaging the veins containing the radiographic medium to obtain a map of the vein and the surrounding connected venous structure, including branches of the vein.

In yet another embodiment, the invention provides methods for geographically mapping a venous structure of the heart utilizing an invention catheter by inserting the catheter into a vein of the heart; occluding the blood flow in the vein; injecting a radiographic medium into the vein, wherein the occlusion of the blood flow causes retrograde flow of the blood and the dye into the surrounding connected venous structure of the vein, including branches of the vein; and imaging the veins containing radiographic medium to obtain a map of the vein and the surrounding connected venous structure, including branches of the vein.

In still another embodiment, the invention provides methods for locating an aberrant electrical conduction pattern in the heart using an invention catheter that has two or more electrodes in the distal end portion of the body by inserting a guidewire into one of the lumens of the invention catheter to form a guidewire-containing balloon catheter; percutaneously inserting the guidewire-containing balloon catheter into a vein of the heart; inflating the balloon to occlude the blood flow in the vein; injecting a radiographic dye into the vein through a lumen of the guidewire-containing balloon catheter, wherein the occlusion of the blood flow by the balloon causes retrograde flow of the blood and the dye into the surrounding connected venous structure of the vein, including branches of the vein; and imaging the vein and connected venous structure containing the radiographic dye so as to obtain a map of the vein and the surrounding connected venous structure, including branches of the vein.

In yet another embodiment of the invention, methods are provided for locating an aberrant electrical conduction pattern in the heart of a subject by inserting a guidewire into one of the lumens of the balloon catheter of claim 10 to form a guidewire-containing balloon catheter, percutaneously inserting the guidewire-containing balloon catheter into a vein of interest in the heart of the subject; inflating the balloon to occlude the blood flow in the vein; injecting a radiographic dye into the vein through a lumen of the guidewire-containing balloon catheter, wherein the occlusion of the blood flow by the balloon causes retrograde flow of the blood and the dye into the surrounding connected venous structure of the vein, including branches of the vein; imaging the veins containing radiographic dye to obtain a map of the vein and the surrounding connected venous structure, including branches of the vein; using the electrodes of the guidewire-containing balloon catheter to obtain a map of the electrical conduction pattern of at least one of area selected from the coronary sinus, coronary sinus ostium, pulmonary vein, branches of the coronary sinus, branches of the pulmonary vein and any other cardiac chamber; and determining a location having an aberrant electrical conduction pattern in the heart of the subject from the map of the electrical conduction pattern.

### BRIEF DESCRIPTION OF THE DRAWINGS

FIG. 1 is a drawing showing a balloon catheter of the invention.

FIG. 2 is drawing in cross-section of the body of the balloon catheter of the invention, showing three lumens therein.

FIG. 3 is a schematic illustration of the balloon catheter of the invention inserted in the coronary sinus of the heart. In the illustration, the balloon of an invention catheter is shown as inflated. The pulmonary artery (PA) of the heart can also be seen in this illustration.

FIG. 4 is a schematic illustration of the balloon catheter of the invention inserted in the pulmonary vein of the heart. Also shown in this illustration are the right atrium (RA), left atrium (LA), aortic valve (AV) and tricuspid valve (TV).

It is noted that the FIGS. 3 and 4 are not intended to be a fully detailed representation of the heart, rather the drawings are presented in an anatomically simplified manner in order to emphasize the features of the invention.

FIG. 5 is a drawing showing a close up view of the proximal end of the balloon catheter of the invention in one embodiment, where the port of entry of one lumen is fitted with a connector, which is connected to a device for monitoring electrical impulses.

FIG. 6 is a drawing showing a close up view of the distal end of the balloon catheter of the invention in one embodiment, where the catheter has two pair of electrodes on the distal end.

FIG. 7 is a drawing of the balloon catheter of the invention in one embodiment, where the catheter has one pair of electrodes at the tip, connected by a lead, running through a lumen, to the device for monitoring electrical impulses.

FIG. 8 is a drawing of the balloon of the balloon catheter of the invention. FIG. 8a shows the balloon in an uninflated, pleated state and FIG. 8b shows the balloon in an inflated state.

### DETAILED DESCRIPTION OF THE INVENTION

The invention catheter is for use in evaluation of coronary sinus or pulmonary vein venography to further elucidate the anatomy of the structures. A catheter of the present invention provides advantages over catheters previously used, in that the invention catheter does not use electrical activation, the catheter does not have a sheath and that the balloon, when inflated, completely occludes the vessel, and causes a retrograde flow of any injected media into the vessel, the branches and surrounding connected structures thereof.

“Venography” as used herein is a test that provides an image of the veins after a radiopaque substance, such as a radiographic dye, is injected into the subject's vein. The test is an invasive insertion of an invention catheter, in which a radiographic substance is injected into a vein in which the catheter is positioned against the blood flow of the vein. The inflated balloon of the catheter occludes the vein and causes retrograde flow into the vessel, the branches and surrounding connected venous structures thereof. Venography can be used to develop a geographical map of the surrounding connected venous structure, including the vein into which the radiopaque or radiographic substance is injected and those veins that are connected, and to view blood flow, blood clots, obstructions, vein defects, inflammation or tumors.

“venous structure” as used herein refers to the network of veins and branches thereof within the heart. A venous structure contains a vein and veins fluidly connected to that vein. Such veins may include, but are not limited to, the pulmonary vein, the coronary sinus, the veins of the left ventricle and branches thereof.

“Radiographic substance” as used herein indicates a substance or contrast medium that is opaque to x-rays and therefore allows radiographic definition of the size shape or location of the vessel in which it is injected. “Radiographic substance” as used herein can also refer to substances that are suitable for imaging by methods other than x-ray and include, but are not limited to, radiographic dye, contrast media, and liposomes containing such a radiographic dye or contrast medium, and the like.

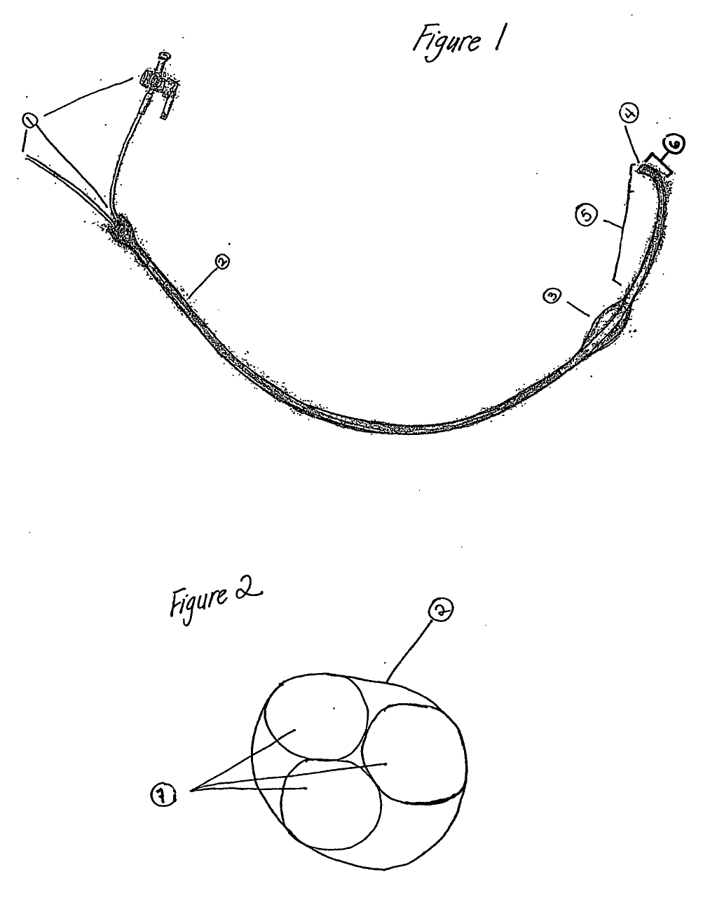

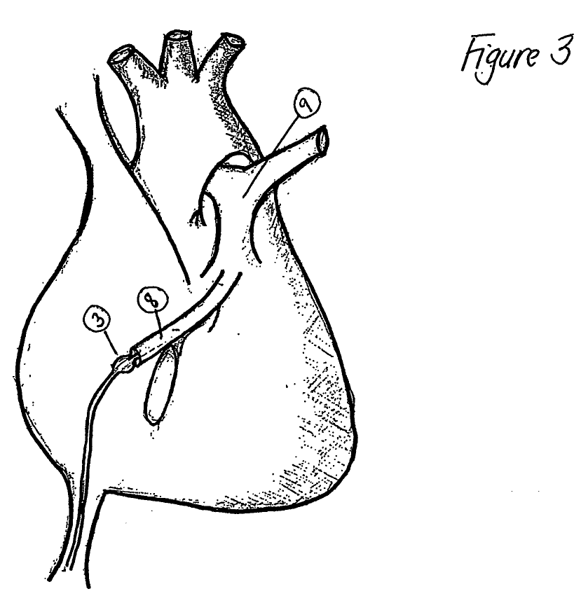

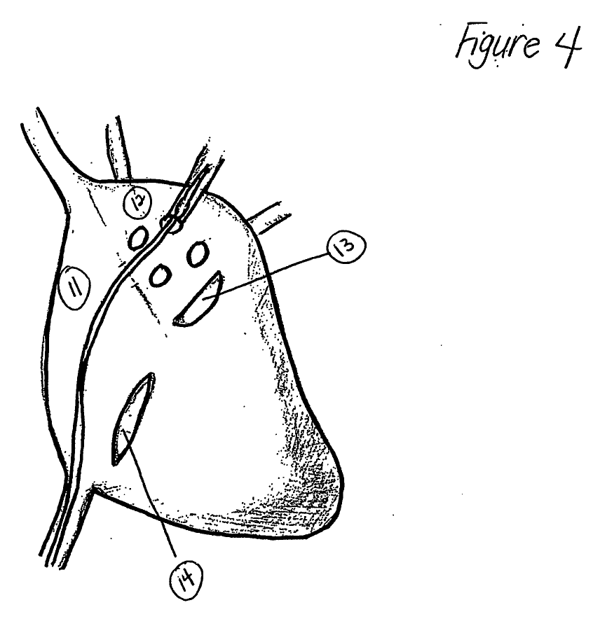

In one embodiment the invention provides balloon catheters with a flexible elongate body and a distal portion comprising curved portion and a hooked portion at the extreme distal end of the distal portion. Additionally, the catheters have at least three lumens running lengthwise within the body, each with a port of entry at the proximal end and, at least one of the lumens has a port of exit at the distal end. Further, the catheters comprise an inflatable balloon located 2 to 4 centimeters proximally of the distal end of the distal portion of the body (i.e., from the curved portion thereof). An illustration of the catheter can be seen in FIG. 1, with the elongate body 2, ports of entry into the lumens 1, balloon 3, port or ports of exit 4, distal end portion 5 of the elongate body and hooked portion 6 at the extreme distal end of the distal end portion. Up to 10 pairs of electrodes may optionally be placed on the distal portion 5, usually spaced apart at intervals of about 1 mm to about 2 mm along the curved portion thereof. FIG. 2 shows a cross section of the body 2 of a balloon catheter of the invention.

The invention catheters may have a body of any convenient length, for example, from about 80 to about 160 centimeters in length, from end to end. The body may be constructed of polyurethane, though one of skill in the art will know of additional materials for the catheter body. In one embodiment, the body of the catheter is about 140 centimeters.

As used herein, “French” is a unit of measurement in the medical arts used to measure the diameter of a catheter. For example, it is known in the art that 3 French is approximately millimeter. The invention catheter has an outside diameter of about 5 to about 7 French or about 1.667 to about 2.333 millimeters or about 0.066 to about 0.092 inches.

As shown in FIG. 1, a catheter of the invention has a distal portion comprising a curved portion with a hooked portion on the extreme distal end. The hooked portion is a bending or increased curvature of the end of the distal end portion to form the “hook,” as compared to the remainder of the distal portion. While both the curved portion and the hooked portion of the distal portion are curved, the hooked portion has a curvature that is greater than the curvature of the curved portion. Therefore a hook is always present at the extreme distal end of the catheter. In one embodiment, the curved portion has a curvature of from about 30 degrees to about 80 degrees, while the hooked portion may have a curvature of from about 40 degrees to about 90 degrees, for example about 65 degrees. The hooked portion is about 2 to about 5 mm long from tip to base where it adjoins the curved portion. The hooked portion on the distal end of the catheter is used to assist the cardiologist in the difficult task of inserting the extreme distal end of the catheter into the coronary sinus ostium of the heart. As is known to those of skill in the art, the coronary sinus extends from the os leftward in the atrioventricular groove around the left lateral border of the heart and ends in an anterior location. The coronary sinus opens into a variable number of branches (usually 3-4), which project at approximately 90-degree angles from the body of the coronary sinus inferiorly down over the epicardial surface of the left ventricle. These branches, although difficult to access, are suitable for placement of permanent pacemaker leads in order to pace the left ventricle. Due to its anatomical geography, access to the coronary sinus with a standard catheter is difficult. The presence of the hooked portion on the invention catheter enhances access of the extreme distal end of the catheter into the coronary sinus via the coronary sinus ostium.

The balloon of the invention catheter surrounds at least a portion of the circumference of the catheter and the distal end of the balloon is located so as to allow for administration of media to the subject when the balloon is either inflated or deflated. For example the distal end of the balloon can be spaced 2 to 4 centimeters proximally from the curved portion of the distal portion of the body. Inflation of the balloon controls the administration of the media so that the media cannot flow into the section of the vessel occluded by the inflated balloon. This occlusion will cause retrograde flow (i.e. against the normal flow of the blood) of the administered media into the venous structure, such as the tributary veins that lead into the main vein or chamber. For example, in an invention catheter intended for insertion through the ostium of the coronary sinus, the balloon is sized to lodge in and block the ostium and strategically located along the exterior of the catheter body to simultaneously allow injection of a radiopaque medium into the coronary sinus.

A catheter of the invention contains one or more lumens throughout the length of the body. In one embodiment the catheter has at least three lumens running lengthwise within the body. The lumens of the invention are independently about 0.025 to about 0.038 centimeters in internal diameter, for example about 0.032 centimeters in internal diameter. A cross-section of the catheter body 2 of FIG. 1 is shown in FIG. 2 with three lumens 6. It can be seen in FIG. 2 that the lumens are independent and can encompass most of the space inside the hollow body of the invention catheter. Each of the lumens has a port of entry at the proximal end and at least one lumen has a port of exit at the distal end for the administration of a drug, medication or radiopaque substance to a subject. One or more ports of entry may be individually fitted with a liquid connector adapted for connection to a fluid source, such as a fluid source for introducing into the lumen a radiopaque fluid for geographical mapping or an air source for inflation of the balloon. At least one of the lumens opens at its distal end into the interior of the balloon so that an expansion fluid can be introduced into the balloon. FIG. 7 shows the lumen opening 19 into the balloon 3. Thus, where one or more ports of entry are used for inflation of the balloon, a media for inflation of the balloon is introduced through the port of entry into the lumen and the distal end of the lumen is in fluid communication with the interior of the balloon. In order to inflate the balloon, a gas or fluid, such as air from a fluid source, passes through the lumen into the interior space of the balloon. If the balloon is inflated before the media is administered and remains inflated during the geographical mapping phase of a procedure, the media will flow away from the point where the catheter was inserted, so that a radiographic dye or similar radiopaque substance will flow into smaller connecting branches of the vein to allow for radiographic mapping of the geographic structure of the venous system.

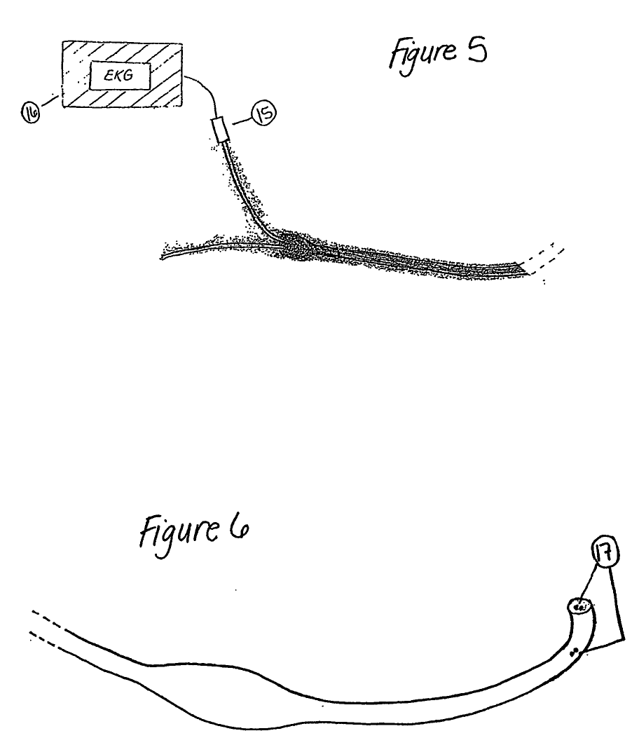

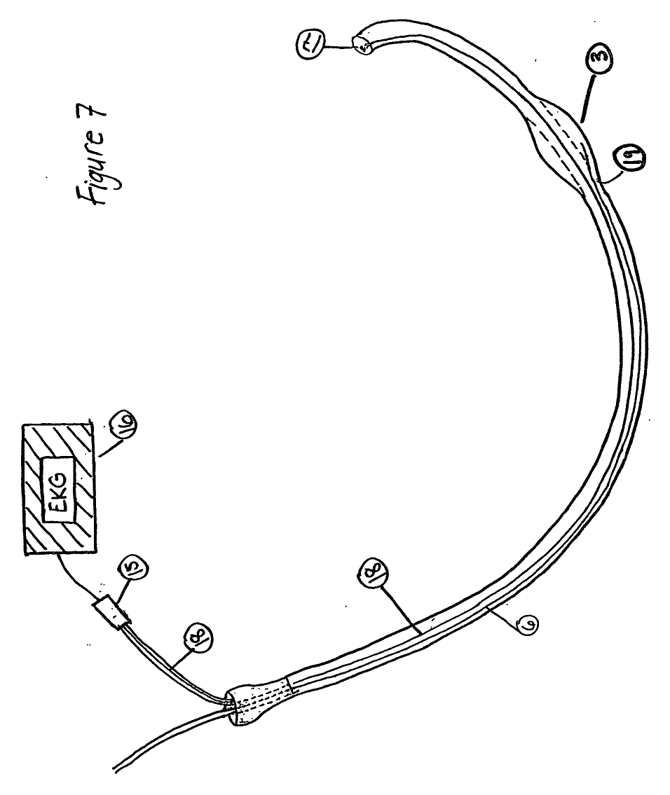

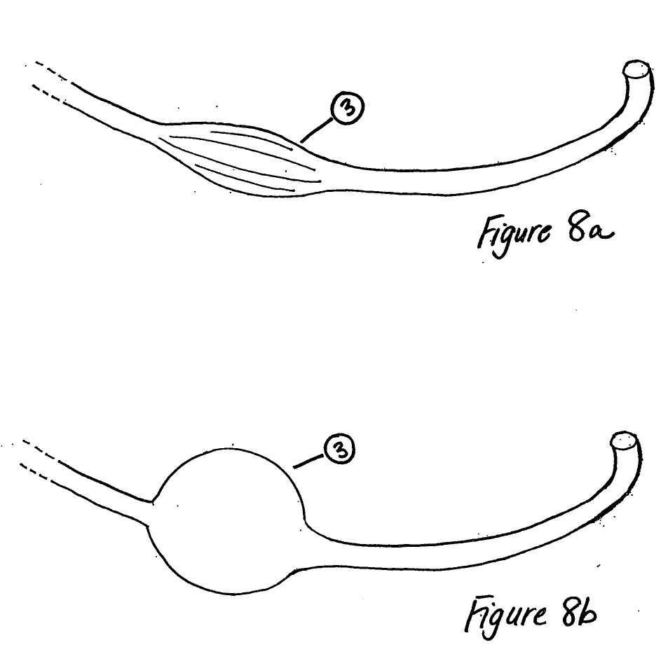

In embodiments of the invention catheter comprising external electrodes, at least one of the lumens is sized for holding lead wires running therethrough and has a port of entry fitted with an electrical connector for establishing electrical connection between the lead wires and a device for monitoring electrical impulses from the electrodes on the external surface of the catheter body, such as an electrocardiogram (EKG) or any other such device as is known to one of skill in the art. Additionally, one of the ports of entry may be used for the insertion of a guidewire to guide the catheter during insertion into the heart. An illustration of such embodiments can be seen in FIGS. 5, 6 and 7. FIG. 5 shows an embodiment where a port of entry is fitted with an electrical connector 15, further connected to an EKG 16. FIG. 6 shows an embodiment where the catheter contains two pair of electrodes 17. FIG. 7 shows an embodiment where the one pair of electrodes 17 is connected by a lead wire 18 to an electrical connector 15, which establishes an electrical connection between the lead wires and an EKG 16.

In one embodiment of the invention, the balloon of the catheter is pleated when not inflated so that the balloon lies close along the exterior of the catheter body when uninflated, to facilitate insertion of the catheter. These pleats allow for expansion to a larger volume when the balloon is inflated so that the balloon, when inflated, forces retrograde flow of an injected substance into the vein in which the catheter is inserted and into the surrounding connected venous structure, preventing and injected the drug or dye from traveling in the normal direction of blood flow. This retrograde flow of drug or dye allows for subsequent imaging of that vein and the surrounding connected venous structure into which a radiopaque medium has been caused to flow. The balloon is sized to fully occlude the venous structure into which it is to be inserted. Those of skill in the art can readily determine appropriate dimensions for a balloon to be used to block any particular venous structure. For example in one embodiment of the invention, when the balloon is inflated, it has a diameter of about 5 mm to about 10 mm and a volume of about 1 cc to about 4 cc, for example, a volume of about 2 cc.

Optionally, a catheter of the invention may also possess at least one bipolar pair of electrodes located externally on the body, for example on the distal end portion of the body. The electrodes may be placed in the distal 10 cm of the body. In another embodiment of the invention, the catheter may have as many as 10 pairs of electrodes. The electrodes are located on the distal portion, usually on the curved portion rather than the hooked portion, and are spaced about 1 mm to about 2 mm apart. However, where the invention catheter has electrodes on the distal portion, they may be located anywhere in that portion, except on the balloon.

The invention provides methods of mapping one or more venous structures of the heart, particularly those in the left ventricle. As set forth above, the heart contains an extensive venous system. Major veins of the heart include the coronary sinus and the pulmonary vein. The invention provides methods of mapping these major cardiac veins, the branches of the veins, the paths of the veins and branches, the orifices of the venous system and the anatomy of the same. All of these may make up the venous structure of the heart. The invention comprises inserting a catheter into a vein of the heart to be mapped, such as the coronary sinus, a pulmonary vein or branches thereof. The catheter is inserted against the blood flow so as to occlude blood flow in the vein. For example, a balloon on the exterior of the catheter can be inflated to occlude the vein at the entry into the vein, while a radiopaque substance, such as a radiographic dye, is injected into the vein and allowed to flow in a retrograde manner into the connecting venous structure, including branches of the vein. The veins containing the radiographic dye are then imaged to obtain a geographic map of the connecting venous structure, including any branches of the vein. By this method, mapping of the coronary sinus, coronary sinus ostium, the pulmonary vein, branches of the coronary sinus, branches of the pulmonary vein and any other cardiac chamber will be performed. Preferred catheters for use in the invention methods for mapping venous structures as described herein are the invention catheters.

Initial venous access is performed as is known to those of skill in the art. The invention catheter can be delivered to the vein of interest using an introducing sheath in any central vein. Central veins for venous access include, but are not limited to: femoral, internal jugular of subdlavian veins.

Another embodiment, the invention provides methods for geographic mapping of a venous structure of the heart by inserting a guidewire into one of the lumens of a balloon catheter and percutaneously inserting the catheter into a vein of the heart. Alternatively, the catheter can be inserted using an introducing sheath. Once in place, the balloon of the catheter is inflated to occlude blood flow in the vein of interest. A radiopaque substance, such as a radiographic dye, is then injected into the vein through a lumen of the catheter and allowed to flow in. retrograde fashion into surrounding connected venous structure. Imaging of the veins is then performed to obtain a map of the vein and the surrounding connected venous structure, including branches of the vein. In the methods of the invention, the venous structure to be mapped may include, but is not limited to, the coronary sinus, a pulmonary vein and branches of the same.

As the blood vessel and the surrounding connected venous structure become filled with the injected radiopaque substance, imaging of the venous structure is possible. Imaging, as used in the present invention may include, but is not limited to x-ray, fluoroscopy, computed tomography (CT), or radiation detection. One of skill in the art will know of and can apply other endocardial imaging techniques to the methods of the invention. Preferably, any occlusion of the veins of the heart by an invention catheter is as brief as possible although the time required will vary depending on a number of factors, the time required to thread the catheter into the vein(s) of interest in the subject's heart taking into account any anomalies in the structure of the subject's heart, the number of veins to be imaged, and the like.

Optionally, as described herein, the catheter may contain electrodes on the distal end of the body, for example from one to 10 pairs of bipolar electrodes. Where the electrodes are present, the geographic map of the venous structure obtained by imaging the radiographic dye in connecting venous structures may be supplemented with a map of the electrical conduction pattern of the venous structure using the same catheter. The electrical map can be obtained by using electrodes on the surface of the catheter, with leads to a device for monitoring electrical impulses from the electrodes, such as an electrocardiogram (EKG) or any other such device as is known to one of skill in the art, to determine the precise location of electrical currents in the venous structure of interest. Alternatively, the technique may be used to obtain a map of the electrical conduction pattern of one or more-venous structures selected from the coronary sinus, coronary sinus ostium, pulmonary vein, branches of the coronary sinus, branches of the pulmonary vein and any other cardiac chamber.

In still another embodiment, the invention provides a method of locating an aberrant electrical conduction pattern in the heart using a guidewire-containing balloon catheter having external distal electrodes, which catheter is percutaneously inserted into the heart. The catheter can optionally be inserted using an introducing sheath. Once in place, the balloon of the catheter is inflated to occlude the blood vessel of interest, forcing retrograde blood flow. A radiopaque substance, such as a radiographic dye, is injected through a lumen of the catheter and the dye is carried with the retrograde blood flow. Imaging of the veins is then performed to obtain a geographical map of the vein and the surrounding connected venous structure, including branches of the vein. The map may contain, but is not limited to, a venous structure of one or more of the coronary sinus, coronary sinus ostium, pulmonary vein, branches of the coronary sinus and branches of the pulmonary vein. The electrodes of the catheter are used to obtain an electrical map of the one or more venous structures. By an analysis of the resulting geographic and electrical maps, a location in the heart having an aberrant electrical conduction pattern can be determined. The invention methods are particularly useful for locating aberrant electrical conduction patterns in the right and left ventricles of the subject, such as necessitate bi-ventricular pacing.

In the imaging of the venous structures having an injected radiopaque substance therein, as described herein, imaging techniques that may be employed include, but are not limited to, x-ray, fluoroscopy, computed tomography (CT), or radiation detection. One of skill in the art will know of and can apply other endocardial imaging techniques to the methods of the invention. Preferably, any occlusion of the veins of the heart by an invention catheter is as brief as possible.

In yet another embodiment of the invention methods, the map of the electrical conduction pattern of one or more venous structures and/or determination of a location of aberrant electrical conduction patterns in a subject is used to facilitate implanting of one or more pacemaker leads. In one embodiment, the one or more pacemaker leads are implanted while the catheter remains in the vein by inserting the one or more pacemaker leads through additional percutaneous opening(s) made in the subject (i.e, a percutaneous opening other than the percutaneous opening made to insert the catheter). In this embodiment it is possible to continue imaging while the pacemaker lead is implanted. Alternatively, the pacemaker lead can be implanted through the same percutaneous opening in the subject as is used to introduce the catheter. The invention methods may further comprise implanting a pacemaker.

A pacemaker implanted by the methods of the invention may be used in a method of traditional pacing or in a method of biventricular pacing, where the pacemaker has leads that stimulate both the right and left regions of the heart.

Although the invention has been described with reference to the above description, it will be understood that modifications and variations are encompassed within the spirit and scope of the invention.. Accordingly, the invention is limited only by the following claims.

What is claimed is:

1. A balloon catheter comprising:
a flexible elongate body having a proximal portion and a distal portion, wherein the distal portion includes a curved portion terminating in a hooked portion at the extreme distal end, wherein curvature of the hooked portion is greater than curvature of the curved portion;
at least three lumens running lengthwise throughout the body, each having a port of entry at a proximal end thereof;
an inflatable balloon located on the exterior of the body with a distal end of the balloon terminating about 2 to 4 centimeters proximally of the curved portion of the distal portion of the body; and
a port of exit located at a distal end of at least one of the lumens.

2. The balloon catheter of claim 1, wherein the body is about 80 to about 160 centimeters in length.

3. The balloon catheter of claim 1, wherein the body is about 5 to about 7 French in diameter.

4. The balloon catheter of claim 2, wherein the curvature of the curved portion is from about 30 degrees to about 80 degrees.

5. The balloon catheter of claim 4, wherein curvature of the curved portion is about 30 degrees.

6. The balloon catheter of claim 5, wherein curvature of the hooked portion is from about 40 degrees to about 90 degrees.

7. The balloon catheter of claim 6, wherein curvature of the hooked portion is about 65 degrees.

8. The balloon catheter of claim 1, wherein the lumens are independently about 0.025 to about 0.038 centimeters in diameter.

9. The balloon catheter of claim 1, wherein the port of entry to at least one of the lumens comprises a connector selected from the group consisting of connectors for connection of the electrodes to a device for monitoring electrical impulses, for inflation of the balloon and for introduction of media to the lumen.

10. The balloon catheter of claim 9, wherein the connector is for monitoring electrical impulses and the catheter further comprises at least one pair of electrodes located externally on the body and distally from the distal end of the balloon.

11. The balloon catheter of claim 10, wherein the electrodes are located on the curved portion of the body distally from the distal end of the balloon.

12. The balloon catheter of claim 9, further comprising leads running through a lumen and connected between the electrodes and the connector for connection of the electrodes to a device for monitoring electrical impulses.

13. The balloon catheter of claim 11, wherein the device for monitoring electrical impulses is an electrocardiogram (EKG) machine.

14. The balloon catheter of claim 9, wherein at least one of the lumen has a connector for inflation of the balloon and the distal end of the lumen opens into an interior of the balloon.

15. The balloon catheter of claim 9, wherein at least one of the lumen is sized for introduction of a guidewire through the lumen.

16. The balloon catheter of claim 10, wherein a single pair of electrodes is located externally on the curved portion of the distal portion of the body.

17. The balloon catheter of claim 10, wherein two pairs of electrodes are located externally along the curved portion of the distal portion of the body.

18. The balloon catheter of claim 10, wherein, from three to ten pairs of electrodes are located externally along the curved portion of the distal portion of the body.

19. The balloon catheter of claim 1, wherein the balloon is sized to expand to a volume of about 1 cc to about 4 cc when inflated.

20. The balloon catheter of claim 1, wherein the balloon has a diameter of about 5 to about 10 millimeters when inflated.

21. The balloon catheter of claim 1, wherein the balloon is pleated when not inflated.

22. The balloon catheter of claim 13, wherein the port of exit is suitable to introduce a drug or radiopaque or radiographic medium to a subject.

23. A method of mapping a venous structure of the heart, comprising:
a) inserting a catheter into a vein of the heart;
b) occluding the blood flow in the vein;
c) injecting a radiographic medium into the vein, wherein the occlusion of the blood flow causes retrograde flow of the blood and the dye into the surrounding connected venous structure of the vein, including branches of the vein; and
d) imaging the veins containing radiographic medium to obtain a map of the vein and the surrounding connected venous structure, including branches of the vein.

24. The method of claim 23, wherein the imaging comprises x-ray imaging, fluoroscopy, computed tomography (CT) or use of a radiation detector.

25. The method of claim 23, wherein the venous structure is the coronary sinus or a pulmonary vein.

26. The method of claim 23 further comprising measuring the electrical conduction pattern of the venous structure and mapping the electrical conduction pattern.

27. A method of mapping a venous structure of the heart, comprising:
a) inserting a guidewire into one of the lumens of the balloon catheter of claim 1 to form a guidewire-containing balloon catheter;
b) percutaneously inserting the guidewire-containing balloon catheter into a vein of the heart;
c) inflating the balloon to occlude the blood flow in the vein;
d) injecting a radiographic dye into the vein through a lumen of the guidewire-containing balloon catheter, wherein the occlusion of the blood flow by the balloon causes retrograde flow of the blood and the dye into the surrounding connected venous structure of the vein, including branches of the vein; and
e) imaging the vein and connected venous structure containing the radiographic dye so as to obtain a map of the vein and the surrounding connected venous structure, including branches of the vein.

28. The method of claim 27, wherein imaging the veins containing radiographic dye is performed by x-ray, fluoroscopy, computed tomography (CT) or radiation detector.

29. The method of claim 27, wherein the venous structure is the coronary sinus or a pulmonary vein.

30. The method of claim 29, wherein the inserting of the guidewire-containing balloon catheter into the coronary sinus is performed by sliding the hooked portion of the catheter through the coronary sinus ostium.

31. The method of claim 27, wherein the catheter has electrodes located externally along the distal end of the body and the method further comprises using the electrodes to obtain a map of the electrical conduction pattern of at least one cardiac chamber or feature.

32. The method of claim 31 wherein the cardiac chamber or feature is selected from the coronary sinus, coronary sinus ostium, pulmonary vein, branches of the coronary sinus, and branches of the pulmonary vein.

33. The method of claim 32, wherein a single pair of electrodes is located externally along the curved portion of the distal portion of the body.

34. The method of claim 32, wherein two pairs of electrodes are located externally along the curved portion of the distal portion of the body.

35. The method of claim 32, wherein from three to ten pairs of electrodes are located externally along the curved portion of the distal portion of the body.

36. A method of locating an aberrant electrical conduction pattern in the heart of a subject, comprising:
a) inserting a guidewire into one of the lumens of the balloon catheter of claim 10 to form a guidewire-containing balloon catheter;
b) percutaneously inserting the guidewire-containing balloon catheter into a vein of interest in the heart of the subject;
c) inflating the balloon to occlude the blood flow in the vein;
d) injecting a radiographic dye into the vein through a lumen of the guidewire-containing balloon catheter, wherein the occlusion of the blood flow by the balloon causes retrograde flow of the blood and the dye into the surrounding connected venous structure of the vein, including branches of the vein;
e) imaging the veins containing radiographic dye to obtain a map of the vein and the surrounding connected venous structure, including branches of the vein;
f) using the electrodes of the guidewire-containing balloon catheter to obtain a map of the electrical conduction pattern of at least one of area selected from the coronary sinus, coronary sinus ostium, pulmonary vein, branches of the coronary sinus, branches of the pulmonary vein and any other cardiac chamber; and
g) determining a location having an aberrant electrical conduction pattern in the heart of the subject from the map of the electrical conduction pattern.

37. The method of claim 36, wherein the imaging of the veins containing radiographic dye is performed by x-ray, fluoroscopy, computed tomography (CT) or radiation detector.

38. The method of claim 36, further comprising using the map of the electrical conduction pattern of the at least one area to determine a location for placement of a pacemaker in the subject.

39. The method of claim 38, further comprising implanting a pacemaker lead into the subject in the location while the guidewire-containing balloon catheter remains in place.

40. The method of claim 39, wherein the implantation of the pacemaker lead comprises introducing the pacemaker lead through a different percutaneous opening in the subject than the opening through which the guidewire-containing balloon catheter was percutaneously inserted.

41. The method of claim 39, wherein the implantation of the pacemaker lead comprises introducing the pacemaker lead through the same percutaneous opening as the opening through which the guidewire-containing balloon catheter was percutaneously inserted.

42. The method of claim 36, wherein the balloon catheter is inserted into the coronary sinus of the coronary artery of the heart and is inflated to occlude the coronary sinus ostium.

43. The method of claim 42, wherein the inserting comprises using the hooked portion of the catheter body to gain entry into the coronary sinus through the coronary sinus ostium.

44. The method of claim 42, wherein the location is the left ventricle and the implanting of the pacemaker lead is in the left ventricle of the heart.

45. The method of claim 44, further comprising attaching at least one of the pacemaker leads to the pacemaker so as to accomplish biventricular pacing.
